(19) United States
(12) Patent Application Publication
(10) Pub. No.: US 2015/0332379 A1
(43) Pub. Date: Nov. 19, 2015

(54) SUBSCRIPTION SERVICE FOR ELECTRONIC CIGARETTES

(71) Applicant: LOEC, Inc., Greensboro, NC (US)

(72) Inventor: Ramon Alarcon, Los Gatos, CA (US)

(21) Appl. No.: 14/711,580

(22) Filed: May 13, 2015

Related U.S. Application Data
(60) Provisional application No. 61/992,679, filed on May 13, 2014.

(51) Int. Cl. G06Q 30/06; G06Q 10/08; G06F 17/30

(52) U.S. Cl. CPC G06Q 30/0635; G06F 17/30879; G06F 17/30595; G06F 17/30339; G06Q 10/087

(57) ABSTRACT

A model for a subscription service for electronic cigarettes includes an e-Cig retailer with a network computer that is configured to communicate with an e-Cig, an e-Cig charging pack, a mobile phone of an e-Cig user, a subscription database, and/or a manufacturer of e-Cigs.

### CROSS-REFERENCE TO RELATED APPLICATIONS

This application claims priority to U.S. States provisional patent application No. 61/992,679, filed on 13 May 2014, which is hereby incorporated by reference in its entirety, for all purposes, as though fully set forth herein

### FIELD OF THE DISCLOSURE

The present disclosure relates to a system, a method, and a device for retailing electronic cigarettes via a subscription service.

### BACKGROUND OF THE DISCLOSURE

Electronic cigarettes, also known as e-cigarettes (eCigs) and personal vaporizers (PVs), are a popular alternative to traditional tobacco-based cigarettes that must be burned in order to generate smoke for inhalation. Electronic cigarettes provide a vapor for inhalation, but do not contain certain byproducts of combustion that may be harmful to human health. Electronic cigarettes are electronic inhalers that vaporize or atomize a liquid solution (smoke juice) into an aerosol mist that may then be delivered to a user. A typical eCig has two main parts—a housing holding a battery and a cartomizer. The housing holding the battery typically includes a rechargeable lithium-ion (Li-ion) battery, a light emitting diode (LED), and a pressure sensor. The cartomizer typically includes a liquid solution, an atomizer and a mouthpiece. The atomizer typically includes a heating coil that vaporizes the liquid solution.

### SUMMARY OF THE DISCLOSURE

According to an aspect of the disclosure, a model for a subscription service for electronic cigarettes includes an e-Cig retailer with a network computer that is configured to communicate with an e-Cig, an e-Cig charging pack, a mobile phone of an e-Cig user, a subscription database, and/or a manufacturer of e-Cigs. Additional features, advantages, and embodiments of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 shows an example of an electronic article that is constructed according to an aspect of the disclosure.

FIG. 2 shows an example of a charging holder that is constructed according to an aspect of the disclosure.

FIG. 3 shows an example of a conceptual overview of a subscription service system for retailing electronic cigarettes.

### DETAILED DESCRIPTION OF THE DISCLOSURE

The disclosure and the various features and advantageous details thereof are explained more fully with reference to the non-limiting embodiments and examples that are described and/or illustrated in the accompanying drawings and detailed in the following. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

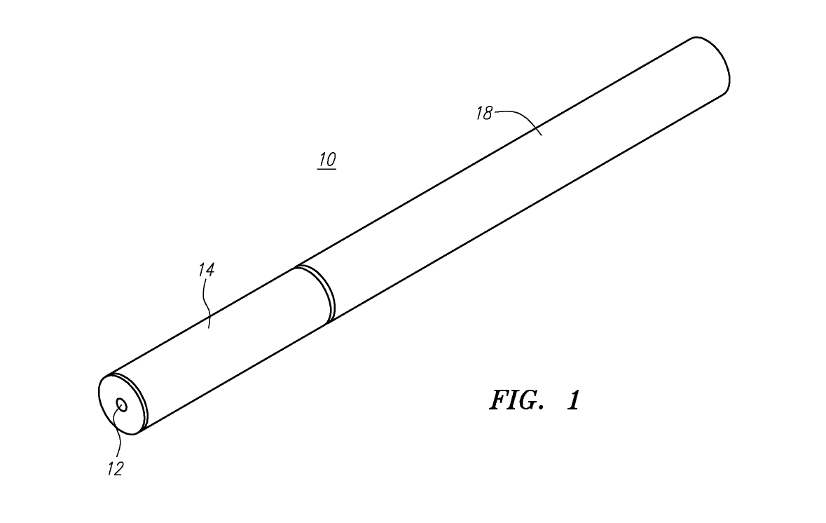

FIG. 1 shows an example of an electronic article 10 according to an aspect of the disclosure. In the instant example, the electronic article 10 comprises an eCig. However, the electronic article 10 may comprise any article that may be charged by an external power supply, such as, e.g., a rechargeable battery, or the like.

The eCig 10 comprises a cartridge 14 and an eCig body 18. The cartridge 14 comprises an opening 12 through which aerosol may be delivered to a user. The cartridge 14 comprises a solution (not shown) and an atomizer (not shown). The solution may include, e.g., a liquid, a gel, a solid, or a gas that comprises molecules (or particles) to be delivered in an aerosol to a user. The eCig body 18 includes a power supply (e.g., a rechargeable Li-ion battery) (not shown) and an LED (not shown). In an alternative embodiment, the cartridge 14 and the eCig body 18 can be combined into a single unit.

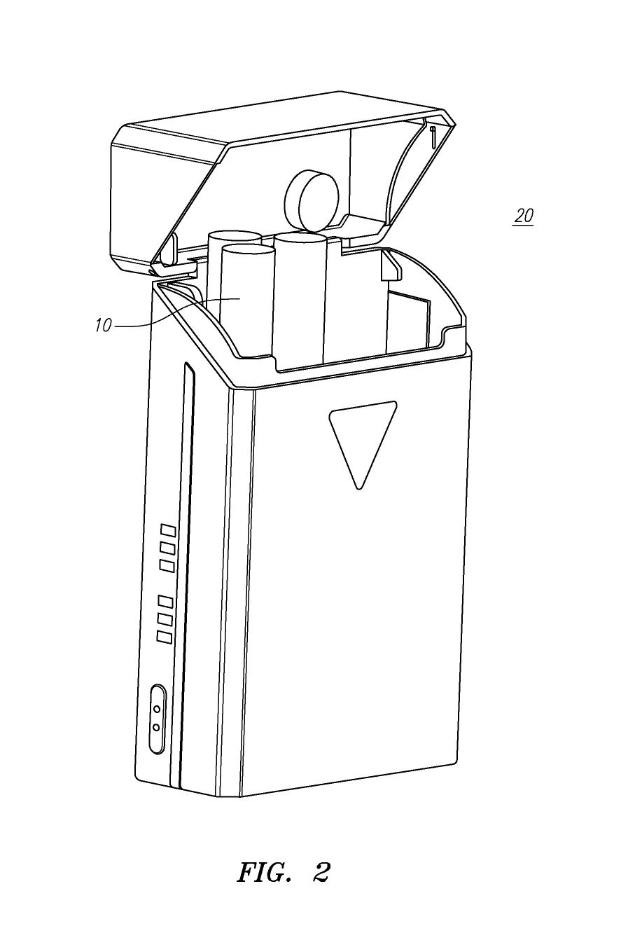

FIG. 2 shows an example of a charging holder (pack) 20. The charging holder 20 in this example comprises a eCig charging pack. The pack 20 comprises one or more regions that are configured to receive the eCig body 18, and/or the cartridge 14, and/or the entire eCig 10. In the example illustrated in FIG. 2, the pack 20 comprises a plurality of regions, each of which is configured to receive a respective eCig 10, or a component of the eCig 10 (e.g., cartridge 14 and/or eCig body 18). The pack 20 comprises a charging device that connects to and supplies a power source to charge the power supply (e.g., Li-ion battery) in the eCig 10, or a component of the eCig 10 (e.g., cartridge 14 and/or eCig body 18).

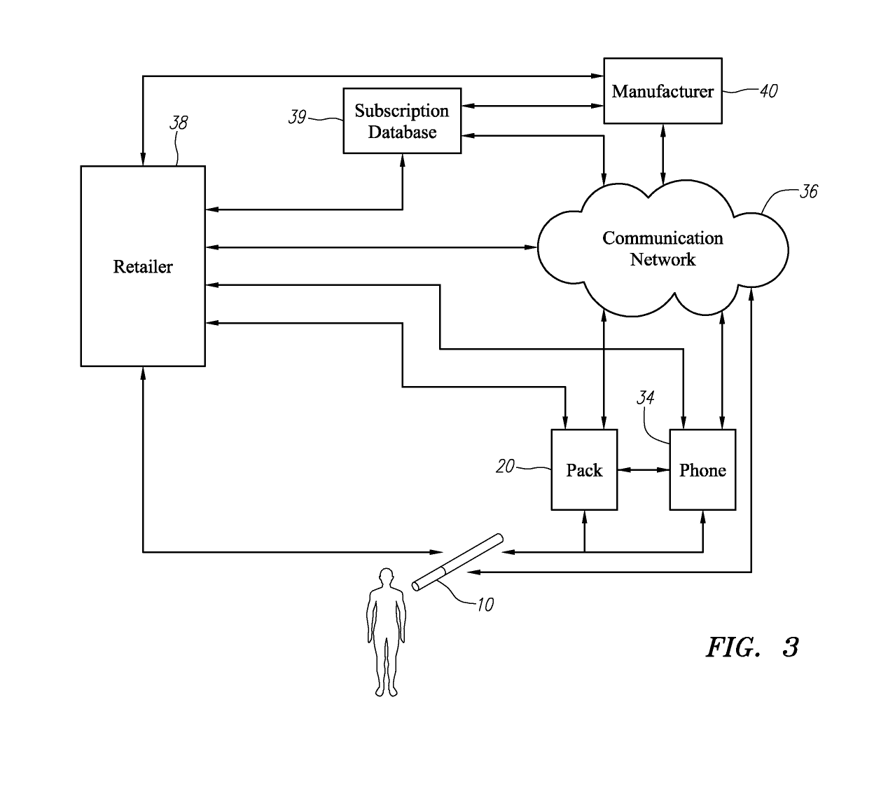

FIG. 3 shows an example of a subscription service system for retailing electronic cigarettes. According to one embodiment of the present disclosure, an e-Cig user can subscribe to a service, such as a refill subscription service, for example. The subscription service can include communication between the user's e-Cig 10, pack 20, or smart phone 34 and a retailer 38, either directly or through a communication network 36 (e.g., LAN, WAN, Internet, intranet, Wi-Fi network, Bluetooth network, cellular network and/or the like). Other devices that can be used instead of or in addition to the smart phone 34 include a mobile phone (not shown), a personal data assistant (PDA) (not shown), a tablet PC (not shown) and/or the like. In some instances, the retailer 38 may communicate with a user's smart phone 34 via the user's pack 20, or vice versa.

The retailer 38 can have a computer network capable of receiving and processing data. The user's e-Cig 10, pack 20, or phone 34 can include a RFID device or barcode, for example, to enable communication with the retailer 38 or network 36. In an example, the retailer 30 can simply scan the RFID or barcode on the eCig 10, pack 20, or phone 34 to obtain data regarding the status of the associated e-Cig 10 or pack 20 (e.g., battery life, level of smoke juice, number of spare cartomizers, the rate at which these supplies are being used, and/or the like). Communication between the user's e-Cig 10, pack 20, or phone 34 and the retailer 38 can also occur via the communication network 36, in which case the retailer 38 can receive data without the need to scan anything.

The subscription service system can also include a separate subscription database 39 that can store data regarding the vamping habits and/or supply status of users. This subscription database 39 can reside on one or more network computers or servers, for example, and can be communicatively linked to both the retailer 38 and the communication network 36. Thus, data regarding users' vamping habits and/or supply status can be stored locally (i.e., by the retailer 38), in a network environment (i.e. in the database), or in both locations.

In an example, when data indicating that a user is low on e-Cig supplies (e.g., pack battery life, smoke juice, customizers, and/or the like) is communicated via the above described channels to the retailer 38, the retailer 38 can send a coupon, a promotional offer, or simply a notice to the user, either directly or via the communication network. The coupon, offer, or notice can be sent to the user's smart phone 34 or pack 20 (e.g. if the pack 20 includes a display screen), for example.

In another example, when data indicating that a user is low on e-Cig supplies (e.g., pack battery life, smoke juice, cartomizers, and/or the like) is communicated via the above described channels to the retailer 38, the retailer 38 or it's computer network can automatically mail the user refill supplies and charge the user's credit card on file. In yet another example, the user can set up recurring shipments of various e-Cig supplies, via the retailer 38 or it's computer network, based on data regarding the user's vaping habits and when he or she will be likely to need refill supplies. Updated or new products can also be sent to users instead of or in addition to refill supplies.

In another embodiment, a retailer can also subscribe to the subscription service. In this case, a network server (not shown) can match up users and retailers, thereby facilitating the sale of e-Cig-related products. For example, if a particular retailer has a large supply of vanilla-flavored smoke juice, and a particular user who uses vanilla-flavored smoke juice is nearly out of smoke juice, the network server could send a coupon for vanilla-flavored smoke juice, redeemable from the retailer, to the user. In another example, the network server could send a coupon or promotional deal to a user who is in the vicinity of a particular retailer that carries vanilla-flavored smoke juice (or any other product). GPS data or other location-based services data (e.g., cellular triangulation or IP address) can be used to match up the retailer and the user.

In yet another embodiment, an e-Cig manufacturer 40 can subscribe to the subscription service, in addition to retailers and users. The manufacturer 40 can have a computer network capable of receiving and processing data, such as sales data for particular retailers or supply status data for particular users, for example. The manufacturer 40 (via its computer network) can be communicatively linked to the communication network 36, and thereby to the subscription database 39, retailer 38, user pack 20, user phone 34, and/or user e-Cig 10. The manufacturer 40 can also be directly linked to the subscription database 39 and the retailer 38. The manufacturer 40 can send (electronically or otherwise) coupons or retail kickbacks, for example, to retailers, based on the data it receives. The manufacturer can also use the data it receives to send coupons or promotions to individual users.

While the disclosure has been described in terms of exemplary embodiments, those skilled in the art will recognize that the disclosure can be practiced with modifications in the spirit and scope of the appended claims. These examples given above are merely illustrative and are not meant to be an exhaustive list of all possible designs, embodiments, applications, or modifications of the disclosure.

What is claimed is:

1. A subscription service system for a user of an electronic smoking device, the system comprising:
at least one of the electronic smoking device or a pack communicatively linked to the electronic smoking device, the pack configured to store or charge the electronic smoking device;
a user mobile device communicatively linked to the at least one of the electronic smoking device or the pack, the user computer configured to store user data;
a subscription database network communicatively linked to at least one of the user mobile device or the at least one of the electronic smoking device or the pack, the subscription database network configured to process the user data and, in response thereto, provide at least one of an automated notification or an automated retail service to the user or to a third party.

2. The subscription service system of claim 1, wherein the at least one of the electronic smoking device or the pack, the user mobile device, and the subscription database network are communicatively linked via at least one of a direct communication channel or a communication network.

3. The subscription service system of claim 2, wherein the direct communication channel comprises a radio-frequency identification (RFID) or a barcode capable of being scanned by a retailer computer; and wherein the RFID or barcode is located on at least one of the electronic smoking device, the pack, or the user mobile device.

4. The subscription service system of claim 1, wherein the user mobile device comprises at least one of a smart phone, a mobile phone, a personal data assistant, or a tablet personal computer.

5. The subscription service system of claim 1, wherein the user data comprises at least one of a supply status of the user, a vaping habit of the user, or a location of the user.

6. The subscription service system of claim 5, wherein the user data comprises at least one of a remaining battery life, a remaining level of smoke juice, a number of spare cartomizers, a rate at which the number of spare cartomizers is being utilized, a rate at which the remaining level of smoke juice is being utilized, and a rate at which the remaining battery life is being utilized.

7. The subscription service system of claim 1, wherein the automated notification comprises at least one of a coupon, a promotional offer, or a message.

8. The subscription service system of claim 1, wherein the automated retail service comprises a recurring or unscheduled refill or replacement purchase of at least one of a cartomizer, a smoke juice, a battery, a new electronic smoking device, or a new pack.

9. The subscription service system of claim 1, wherein the subscription database network is configured to provide the automated notification to at least one of the user mobile device or the pack.

10. The subscription service system of claim 1, wherein the third party is at least one of a retailer or a manufacturer of the electronic smoking device.

11. The subscription service of claim 1, further comprising at least one of a retailer or a manufacturer communicatively linked to the subscription database network.

12. The subscription service of claim 11, wherein the retailer comprises a retail computer network.

13. The subscription service of claim 11, wherein the manufacturer comprises a manufacturer computer network.

14. The subscription service of claim 11, wherein the subscription database network is configured to match the retailer with a user based on at least one of the user data, a location of the user, or an inventory of the retailer.

15. The subscription service of claim 11, wherein the subscription database network is configured to match the manufacturer with at least one of the retailer or a user based on at least one of the user data, a location of the user, or an inventory of the retailer.

16. A method of providing a subscription service to a user of an electronic smoking device, the method comprising:
collecting user data from at least one of the electronic smoking device, a pack configured to store or charge the electronic smoking device, or a user mobile device; and
providing at least one of an automated notification or an automated retail service in response to the user data.

17. The method of claim 16, wherein the user data comprises at least one of a supply status of the user, a vaping habit of the user, or a location of the user.

18. The method of claim 16, wherein providing at least one of an automated notification or an automated retail service comprises matching the user data with data from at least one retailer.

19. The method of claim 18, wherein the data from the at least one retailer comprises inventory data or location data.

20. The method of claim 16, wherein providing at least one of an automated notification or an automated retail service comprises providing at least one of a coupon, a promotional offer, a message, or a recurring or unscheduled refill or replacement purchase of at least a portion of the electronic smoking device or pack.
